(19) United States
(12) Patent Application Publication
(10) Pub. No.: US 2006/0149141 A1
(43) Pub. Date: Jul. 6, 2006

(54) Method of using pressure to determine the positioning of a catheter within a breast duct

(76) Inventor: Ellen Sheets, Concord, MA (US)

Correspondence Address: Theodore Allen, Esq.;Cytyc Corporation, 250 Campus Drive, Marlborough, MA, 01752, US

(21) Appl. No.: 11/297,530

(22) Filed: Dec. 8, 2005

Related U.S. Application Data
(60) Provisional application No. 60/640,708, filed on Dec. 30, 2004.

(51) Int. Cl. A61B 5/00

(52) U.S. Cl. 600/300; 600/573

(57) ABSTRACT

The present invention relates to the field of systems of methods for the positioning of medical devices in body cavities. More particularly, the present invention relates to a system and method that uses pressure to detect the position a catheter within a breast duct.

### FIELD OF THE INVENTION

The present invention relates to the field of systems of methods for the positioning of medical devices in body cavities. More particularly, the present invention relates to a system and method that uses pressure to detect the position a catheter within a breast duct.

### BACKGROUND OF THE INVENTION

The breast is a specialized, glandular structure including a system of complicated breast ducts that radiate from the nipple and that are bound together by fairly dense connective tissue. Each of these breast ducts includes an associated ductal orifice on the surface of a nipple through which ductal fluid may be expressed. Each duct includes a series of successive interlobular branches that drain through the main, lactiferous branch, which terminates and exits the breast at the nipple via the associated ductal orifice. Immediately proximate the ductal orifice, each lactiferous duct includes a lactiferous sinus in which ductal fluid may accumulate. A ductal sphincter resides within the lactiferous sinus and prevents ductal fluid from unintentionally exiting the breast duct through its associated ductal orifice.

Breast cancer is believed to begin in the lining of these breast ducts. For several decades significant members of the medical community dedicated to studying breast cancer have believed and shown that the cytological analysis of cells retrieved from nipple discharge fluid from within breast ducts may provide valuable information leading to identifying patients at risk for breast cancer. Indeed, Papanicolaou contributed to the genesis of such a possibility of a “Pap” smear for breast cancer by analyzing the cells contained in nipple discharge. More recently, cancer specific markers have been detected in ductal fluid obtained by nipple aspiration. However, the retrieval techniques and instruments used by these members of the medical community did not routinely obtain meaningful ductal fluid samples.

In their attempts to retrieve the breast duct fluid sample including ductal epithelial cells, practitioners introduced indwelling hair-like single lumen catheters through the ductal orifice and into the ductal network. This technique, called ductal lavage, is a minimally invasive medical procedure that involves collecting samples of breast ducts cells and then examining the cells under a microscope to determine whether they are normal, atypical, suspicious, or malignant. However, these techniques required that significant, sometimes painful, pressure be created on the nipple surface or along the sides of the breast to overcome the fluid retaining properties of the ductal sphincter. Also, these techniques did not routinely provide meaningful ductal fluid samples with a sufficient number of ductal epithelial cells for a meaningful cellular analysis. These techniques typically caused the recovery of samples with twenty or fewer ductal epithelial cells. Additionally, these techniques did not provide samples with cell clusters of 10 or more cells. As a result, the obtained fluid samples could not consistently provide an accurate indication of whether or not the duct from which they were retrieved included precancerous or cancerous cells. Consistent, meaningful ductal epithelial cell samples have been provided by the medical instrument disclosed in U.S. Pat. No. 6,413,228 to Hung et al. that is hereby incorporated by reference in its entirety.

Other medical instruments, such as those used during galactography, are introduced into the breast duct in order to visually determine the presence of cancerous cells within a breast duct. However, these devices typically extend a significant distance out of the breast duct during the performed procedure. These distances may be twelve inches or greater. As a result, when an operator is not holding the tool, the moment created by the weight and length of the section of the instrument extending out of the duct may cause the indwelling portion of the instrument to engage the sidewalls of the duct, torque and/or kink the duct and distort the nipple. These effects on the duct and nipple may impede the procedure by twisting or crimping the indwelling portion of the instrument, possibly injuring the patient's duct and causing significant discomfort to the patient. As a result, a patient must either endure the pain and discomfort caused by these long instruments or an attendant must constantly support the instrument above the patient during the medical procedure.

Patients with tight ductal sphincters or tortuous ductal orifices may experience difficulties with the lavage procedure due to twisting or crimping the indwelling portion of the catheter, possibly injuring the patient's duct and causing significant discomfort to the patient. Even if the catheter is navigated through tight ductal sphincters and narrow ductal orifices, it is difficult to determine whether the catheter tip is actually residing within the breast duct or is situated against the ductal wall or within the parenchyma of the breast. Thus, it is an object of this invention to provide an improved method for accessing breast duct fluid with minimal discomfort to the patient.

### SUMMARY OF THE INVENTION

According to the invention, this object is achieved in that a medical device (e.g., a catheter) can be placed into a body cavity (e.g., a breast duct) while monitoring the pressure within the body cavity. By monitoring the pressure within the body cavity, the user can determine whether or not the medical device is properly placed within the body cavity.

Thus, in one aspect of the present invention, a method for detecting the placement of a medical device within a body cavity, comprising the steps of: (a) inserting a medical device into a body cavity; (b) monitoring a pressure within the body cavity; and (c) if the pressure monitored in step (b) rises above a predetermined threshold level, providing feedback to the user that the medical device is not properly placed within said body cavity is presented.

In another aspect of the invention, a method for detecting the placement of a medical device within a body cavity, comprising the steps of inserting a medical device into a body cavity and monitoring the pressure within the body cavity and if the pressure monitored remains steady or remains below a predetermined threshold level, feedback is provided to the user that the medical device is properly placed within the body cavity is provided. In yet another aspect of the present invention, the body cavity is a breast duct and the medical device is a catheter.

In still another aspect of the invention, a method for detecting the placement of a medical device within a body cavity, comprising the steps of inserting a medical device into a body cavity which contains a pressure detector, and monitoring a pressure within the body cavity using said pressure detector is provided.

In still yet another aspect of the invention, a method for the detection of a feedback signal from a user input device and discontinuing use of the medical device within a body cavity following detection of the feedback signal is provided.

In still yet another aspect of the invention, a system for the placement of a medical device in a body cavity, the system including; a medical device insertable into a body cavity; a pressure sensor positioned to detect a pressure of the body cavity; and feedback means coupled to the pressure sensor to provide feedback to a user if the pressure monitored by the pressure sensor rises above a predetermined level is provided.

In another aspect of the present invention, a method for detecting the placement of a catheter within a breast duct, comprising the steps of: (a) inserting a catheter into a breast duct; (b) monitoring a pressure within the breast duct; and (c) if the pressure monitored in step (b) rises above a predetermined threshold level, providing feedback to the user that the catheter is not properly placed within said breast duct is presented.

In another aspect of the invention, a method for detecting the placement of a catheter within a breast duct, comprising the steps of inserting a catheter into a breast duct and monitoring the pressure within the breast duct and if the pressure monitored remains steady or remains below a predetermined threshold level, feedback is provided to the user that the catheter is properly placed within the breast duct is provided.

In still another aspect of the invention, a method for detecting the placement of a catheter within a breast duct, comprising the steps of inserting a catheter into a breast duct which contains a pressure detector, and monitoring a pressure within the breast duct using said pressure detector is provided.

In still yet another aspect of the invention, a method for detecting the placement of a catheter within a breast duct, comprising the steps of inserting a catheter into a breast duct and monitoring the pressure within the breast duct and the detection of a feedback signal from a user input device and discontinuing use of the catheter within a breast duct following detection of the feedback signal is provided.

In still yet another aspect of the invention, a method for detecting the placement of a catheter within a breast duct, comprising the steps of inserting a catheter into a breast duct which contains a pressure detector and monitoring the pressure within the breast duct and the detection of a feedback signal from the pressure detector and discontinuing use of the catheter within a breast duct following detection of the feedback signal is provided.

In still yet another aspect of the invention, a system for the placement of a catheter in a breast duct, the system including; a catheter insertable into a breast duct; a pressure sensor positioned to detect a pressure of the breast duct; and feedback means coupled to the pressure sensor to provide feedback to a user if the pressure monitored by the pressure sensor rises above a predetermined level is provided.

### DETAILED DESCRIPTION OF THE INVENTION

The FirstCyte E-Z Microcatheter (Cytyc, Marlborough, Mass.) is a ductal catheter that is placed within a breast duct blindly in order to obtain ductal epithelial cells for a cytologic diagnosis via flushing the duct with a balanced electrolyte solution in a manner known as ductal lavage. Currently, ductal lavage microcatheters consists of a central dilator probe within a plastic catheter that is attached to an F adapter where two separate tubes enter for flushing the catheter and the duct with a balanced electrolyte solution (see U.S. patent application Ser. Nos. 10/746,128, 10/746,950, and 10/746,117, hereby incorporated by reference in their entirety).

One of the persistent difficulties with the ductal lavage procedure is determining whether the catheter tip is residing within the breast duct or whether it has been placed into the substance or parenchyma of the surrounding breast. The result of extra-ductal placement is that no breast duct epithelial cells will be found in the balanced electrolyte solution returning from the catheter flush wash during the lavage procedure.

It is proposed that the breast duct represents a potential body space much like the peritoneal cavity. Upon entry into such spaces via a medical device such as a catheter, the pressure within an externally derived system should drop, or at least not increase appreciably. Such a phenomenon could be described as an opening pressure that could be determined upon placement of the microcatheter by measuring the pressure within the microcatheter system.

The pressure in the system could be measured by the placement of a pressure sensor connected to a pressure gauge on the tube(s) entering the F adapter. A drop or minimal increase in system pressure would indicate that the microcatheter tip was within the ductal space. An increase in pressure would indicate that the microcatheter tip might be against the ductal wall or within the parenchyma of the breast. Readjustment of the microcatheter tip may cause the pressure to drop and allow for continuation of the ductal lavage procedure. If the pressure remained elevated, then the lavage procedure would be terminated.

Determining the pressure within the microcatheter system could decrease the occurrence of insufficient cells for medical diagnosis. Patients who have elevated pressures at the time of lavage could be brought back at an interval for additional attempts at the procedure and prior dilation of the ductal opening with small probe dilators could be utilized to increase the possibility of successful placing of the microcatheter within the breast duct.

The method of lavage may include seating a patient substantially upright in a chair during the lavage procedure, rather than the standard or classic supine (face up) position. Alternatively, the patient may be lavaged in a prone position, face down, with nipples and breast down. The prone face down position takes advantage of gravity and allows the breast ducts to drain into the collection receptacle during the procedure when the outflow port is open. Thus, as discussed previously, the lavaging procedure may include infusing the breast duct with a wash fluid through an open inflow lumen while an outflow lumen is closed; closing the inflow lumen when the duct is filled; squeezing or massaging the breast or both; and opening the outflow lumen to collect the wash fluid.

The cells collected may comprise ductal epithelial cells; the ductal fluid collected may comprise molecular and cellular material. The collected cells and fluid and fluid components may be analyzed. The lavage fluid including the ductal cells may be analyzed for diagnostic purposes. Conditions in a breast milk duct that are desirable to diagnose include a cancer or precancer condition. The precancer condition may include atypical ductal hyperplasia (ADH) or low-grade ductal carcinoma in situ (LG-DCIS). The diagnostic agent may also have the ability to diagnose other breast related conditions, including, e.g. fibrotic, cystic or conditions relating to lactation. Diagnostic agents may be mixed with the ductal fluid (either in the lavage procedure, or after the fluid is collected).

The fluid infused into the duct to lavage the duct may include known, biocompatible fluids. These lavage fluids may include saline, phosphate buffered saline, a nonabsorbable fluid, an isotonic solution, an osmotic solution, a hypotonic solution, and a hypertonic solution. The wash fluid may comprise for example, saline, phosphate buffered saline, a nonabsorbable fluid, an isotonic solution, an osmotic solution, a hypotonic solution, a hypertonic solution. a protein, a colloid, a sugar, a polymer, mannitol, sorbitol, glucose, glycerol, sucrose, raffinose, fructose, lactulose, sodium chloride, polyethyleneglycol (PEG), maltodextrin, dextran (e.g. dextran 70), hydroxyethyl starch, fluid gelatin, a synthetic colloid, an antibody, a binding protein, or albumin.

Any number of alternative combinations could exist for defining the invention, which incorporate one or more elements from the specification, including the description, claims, and drawings, in various combinations or sub combinations. It will be apparent to those skilled in the relevant technology, in light of the present specification, that alternate combinations of aspects of the invention, either alone or in combination with one or more elements or steps defined herein, may be utilized as modifications or alterations of the invention or as part of the invention. It may be intended that the written description of the invention contained herein covers all such modifications and alterations.

1. A method of detecting the placement of a medical device in a body cavity, comprising the steps of: (a) inserting a medical device into a body cavity; (b) monitoring a pressure within the body cavity; and (c) if the pressure monitored in step (b) rises above a predetermined threshold level, providing feedback to the user that the medical device is not properly placed within said body cavity.

2. The method of claim 1, wherein the method further includes the step of, if the pressure monitored in step (b) remains steady or remains below a predetermined threshold level, providing feedback to the user that the medical device is properly placed within the body cavity.

3. The method of claim 1, wherein the body cavity is a breast duct.

4. The method of claim 1 wherein the medical device is a catheter

5. The method of claim 1, including the steps of: further providing a pressure detector, and performing step (b) using said pressure detector.

6. The method of claim 1 wherein the method further includes the step of detecting a feedback signal from a user input device and discontinuing use of the medical device within the body cavity following detection of the feedback signal.

7. A system for detecting the placement of a medical device in a body cavity, the system including; a medical device insertable into a body cavity; a pressure sensor positioned to detect a pressure of the body cavity; and feedback means coupled to the pressure sensor to provide feedback to a user if the pressure monitored by the pressure sensor rises above a predetermined level.

8. The system of claim 7, wherein the body cavity is a breast duct.

9. The system of claim 7, wherein the medical device is a catheter

10. The system of claim 7, wherein the system further includes a feedback means coupled to the pressure sensor to provide feedback to a user if the pressure monitored by the pressure sensor remains the same or decreases below a predetermined level.

11. The system of claim 7, wherein the feedback means is for providing auditory feedback to a user.

12. The system of claim 7 wherein the feedback means is for providing visual feedback to a user.

13. The system of claim 7 wherein the feedback means includes a processor electronically coupled to the pressure sensor, the processor configured to generate a feedback signal in the event the monitored pressure rises above a predetermined level; and a feedback device coupled to the processor, the feedback device responsive to the feedback signal for alerting a user to the position of a medical device in the body cavity.
